(19) United States
(12) Patent Application Publication
(10) Pub. No.: US 2022/0288292 A1
(43) Pub. Date: Sep. 15, 2022

(54) SOLUTIONS FOR CONTINUOUS INFUSION OF A DRUG ADMINISTERED VIA DIALYSIS AND DOSING ALGORITHM THEREFOR

(71) Applicants: DREXEL UNIVERSITY, Philadelphia, PA (US); NEW YORK UNIVERSITY, New York, NY (US)

(72) Inventors: Jeffrey John Cies, Pottstown, PA (US); Arun Chopra, Springfield, PA (US)

(21) Appl. No.: 17/750,547

(22) Filed: May 23, 2022

Related U.S. Application Data
(63) Continuation of application No. 16/489,618, filed on Aug. 28, 2019, now abandoned.
(60) Provisional application No. 62/465,318, filed on Mar. 1, 2017.

(51) Int. Cl. A61M 1/16; A61M 1/34; A61B 5/00

(52) U.S. Cl. CPC A61M 1/1654; A61M 1/1621; A61M 1/3458; A61B 5/4839; B01D 61/24

(57) ABSTRACT

A method of preparing a solution for use during a dialysis, hemodialysis, or Continuous Renal Replacement Therapy (CRRT) treatment of a patient is provided. An antibiotic or other drug is added (48, 50, 52) to a solution and the concentration or dosing of the antibiotic or other drug within the solution is adjusted (72) as required based on a determination (64) of serum level of the antibiotic in a blood sample of a patient.

### CROSS REFERENCE TO RELATED APPLICATIONS

This application is a continuation of co-pending U.S. application Ser. No. 16/489,618 which is a 371 National Stage of International Application No. PCT/US2018/020259, filed Feb. 28, 2018, which claims the benefit under 35 USC § 119(e) of U.S. Provisional Patent Application No. 62/465,318, filed Mar. 1, 2017.

### BACKGROUND

Continuous Renal Replacement Therapy (CRRT), for example, is a form of dialysis for intensive care unit (ICU) patients. Generally, CRRT is used for patients that are too ill to have other forms of dialysis, is performed at bedside in an ICU, and is better tolerated by the patient relative to other dialysis procedures since blood pressure may be relatively low and/or unstable. Typically, a smaller amount of blood is removed from the patient's body using a relatively smaller filter as compared to other dialysis procedures. Thus, CRRT is generally considered a gentler process with slower blood flow and slower removal of waste and extra fluids.

Procedurally, a pump within a hemodialysis machine moves blood from a blood tube connected to the patient into a dialysis filter. The filter cleans the blood removing waste and extra water, and then the cleaned blood is returned to the patient's body via a return blood tube. For CRRT, this process is applied continuously, and treatment may continue (24 hours a day) until the patient's kidneys are able to function on their own or the patient is able to tolerate regular hemodialysis. Patients on CCRT remain in the intensive care unit and are watched closely by nurses and doctors during treatment.

When administering an antibiotic or the like, such as vancomycin, in a pediatric intensive care unit (PICU) or an adult ICU for treatment of invasive infections, such as meningitis, pneumonia, and blood stream infections, current national guidelines recommend a target trough concentration of 15-20 mg/L or an area under the concentration time curve (AUC) to minimum inhibitory concentration (MIC) ratio (AUC:MIC)≥400:1 to maximize efficacy. Infections in both adult and pediatric ICU patients require rapid attainment of therapeutic antimicrobial concentrations to improve morbidity and mortality. Given reduced and variable penetration of vancomycin or like antibiotic drug into organs such as the lungs or central nervous system, higher doses are often required to achieve desired therapeutic targets.

Disease states such as sepsis can significantly alter the pharmacokinetics of many drugs in an ICU setting, including vancomycin, with resultant reduced serum drug concentrations due to increased volume of distribution and compounded by augmented renal clearance. For instance, vancomycin dosing regimens are often unlikely to achieve target concentrations in critically ill septic patients. Furthermore, continuous renal replacement therapy (CRRT) discussed above, commonly initiated in acute kidney injury, also alters drug pharmacokinetics. Thus, there are reports of variable vancomycin clearance during CRRT, and optimal dosing regimen remains unknown.

### SUMMARY

A method of adding a drug or like substance to a solution being prepared for use during hemodialysis, continuous renal replacement therapy (CRRT), or like treatment for administering the drug or like substance to a patient is provided.

### BRIEF DESCRIPTION OF THE DRAWINGS

Various features of the embodiments described in the following detailed description can be more fully appreciated when considered with reference to the accompanying figures, wherein the same numbers refer to the same elements.

FIG. 1 is a diagram showing process steps for selection of a particular drug and a starting concentration for the drug to be administered to the patient according to an embodiment.

FIG. 2 is a diagram showing process steps for a dosing algorithm according to an embodiment.

### DETAILED DESCRIPTION

For simplicity and illustrative purposes, principles of embodiments are described herein by referring primarily to examples thereof. In the following description, numerous specific details are set forth to provide a thorough understanding of the embodiments. It will be apparent to one of ordinary skill in the art that the embodiments may be practiced without limitation to these specific details. In some instances, well known methods and structures have not been described in detail so as not to unnecessarily obscure the embodiments.

“Patient” or “subject” as used herein means a mammalian animal, including a human, a veterinary or farm animal, a domestic animal or pet, and animals normally used for clinical research.

Embodiments disclosed herein may include the use of any continuous infusion drug or like medical substance that may be given in a prolonged or continuous infusion modality. For example, the drug or like medical substance may be an antibiotic, anti-infective, sedative, anti-fungal medication, anti-viral medication, analgesics, sedatives, anti-epileptics, vasoactives, anti-hypertensives, or the like.

Solely for the purpose of providing an example, and not for purpose of limitation, one example of a drug useful in embodiments disclosed herein is vancomycin which is an antibiotic highly effective against gram-positive bacteria, for instance staphylococci. Vancomycin may be administered intravenously in the treatment of severe staphylococcal infections which tend to be resistant to other antibiotics. Continuous infusion (CI) of vancomycin is able to achieve target concentrations more rapidly than intermittent dosing with a reduced incidence of acute kidney injury. While embodiments disclosed herein may refer to vancomycin for purposes of providing an example, the embodiments are not limited to this particular antibiotic, drug, or medical substance.

Accordingly, at least some embodiments disclosed herein relate to continuous infusion of a drug, such as an antibiotic, for instance vancomycin, by mixing the antibiotic into a solution in-vitro which is then provided for use in a CRRT or other hemodialysis procedure. Thus, blood being continuously withdrawn and cleaned by a dialysis filter during such a procedure is simultaneously infused with the drug or antibiotic in vitro from the solution and then returned to the patient. In this manner, therapeutic drug concentrations are able to be more readily achieved and problems experienced during CRRT referenced above, for instance, may be overcome.

Examples of CRRT may include the following procedures: continuous veno-venous hemofiltration (CVVH); continuous veno-venous hemodialysis (CVVHD); and continuous veno-venous hemodiafiltration (CVVHDF). Embodiments disclosed herein may be utilized with any of the above referenced procedures, for instance, for a patient having a documented or suspected gram-positive bacterial infection necessitating the use, for instance, of vancomycin while receiving CRRT. Embodiments disclosed herein may also be utilized with other hemodialysis or dialysis procedures. The administration of continuous infusion vancomycin, for instance, is accomplished according to embodiments disclosed herein by mixing vancomycin in a CRRT or like solution or solutions.

The CRRT or like solution may be prepared at the time it is needed following current standard operating procedures for sterile product compounding or as discussed below in greater detail. The amount of vancomycin, for instance, to be added to the solution is described below in greater detail. Accordingly, this mixed solution is provided for being infused for the duration it is prescribed for CRRT. Vancomycin, for example, is stable with components of the CRRT solution for at least 96 hours. Therefore, the frequency with which each solution bag or like vessel is used does not exceed 96 hours. Other drugs may be stable for less or more time.

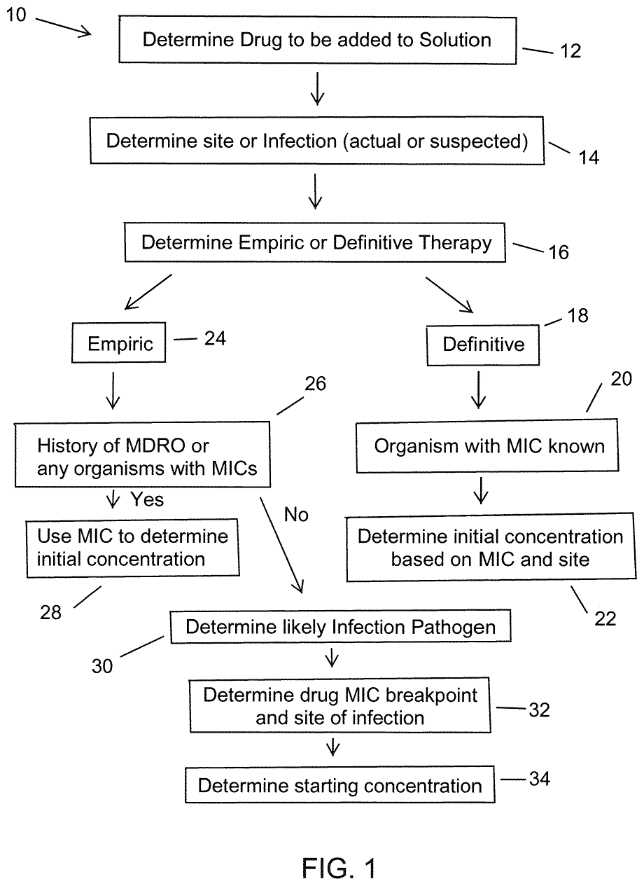

As discussed above, embodiments disclosed herein are not limited to any particular continuous infusion drug. A process 10 of determining a particular drug and a starting concentration of the drug may be as shown in FIG. 1.

After the drug to be used is determined in step 12, and a site of actual or suspected infection within a provided patient is determined in step 14, a decision as to whether the applied therapy is an empiric therapy or a definitive therapy is made in step 16. For a definitive therapy 18, an organism with minimal inhibitory concentration (MIC) is known and identified in step 20 and a determination of the starting concentration is made in step 22 based on MIC and site. For an empiric therapy 24, a decision in step 26 is made as to whether or not a history of multi-drug resistant organism (MDRO) or any organism with minimal inhibitory concentrations (MICs) is known. If so, the MIC is used to determine the starting concentration in step 28. If not, a determination is made concerning the likely infection pathogen in step 30, and the drug MIC breakpoint and site of infection is determined in step 32. From this information, the starting concentration may be determined in step 34.

Solely for purposes of example, vancomycin may be the drug to be administered. It should be understood that the use of vancomycin is only discussed below for purposes of example and that the procedures discussed below could be used with any other type of continuous infusion drug or antibiotic selected, for instance, as a result of the process disclosed relative to FIG. 1. By way of example and not by way of limitation, the drug may include glycopeptide, oxazolidinone, any other anti-infective that displays time dependent anti-infective properties, an anti-viral agent, or any of the drugs referenced above.

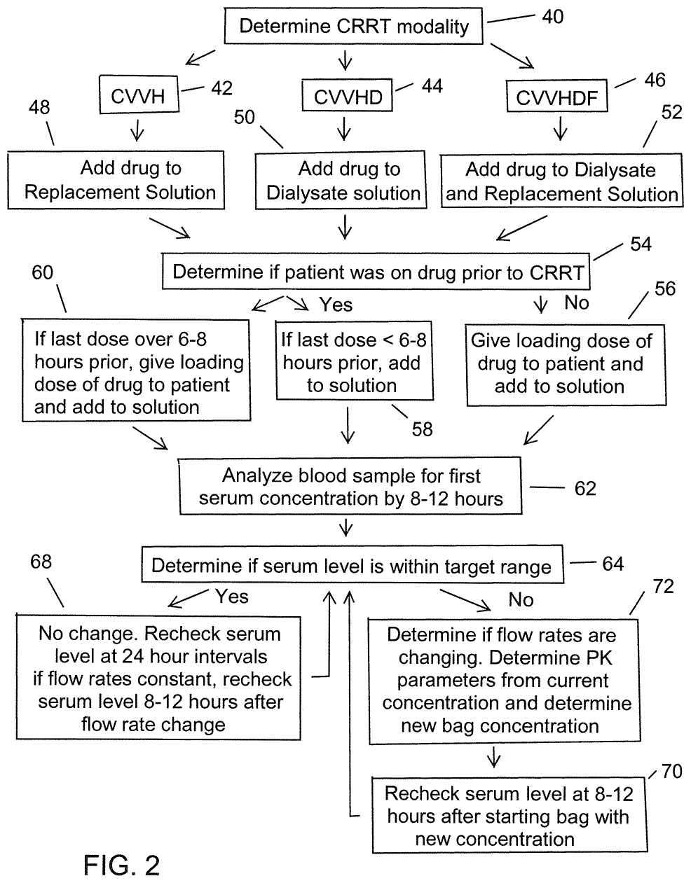

In FIG. 2, after the drug and starting concentration of the drug has been determined, a type of hemodialysis or CRRT may be selected or made known in step 40, such as continuous veno-venous hemofiltration (CVVH) 42, continuous veno-venous hemodialysis (CVVHD) 44, or continuous veno-venous hemodiafiltration (CVVHDF) 46. If CVVH is to be utilized, the drug is added to a replacement solution in step 48. Alternatively, if CVVHD is to be utilized, the drug is added to dialysate solution in step 50, or if CVVHDF is to be utilized, the drug is added to dialysate and replacement solutions in step 52.

A further step 54 requires a determination as to whether or not the drug may have been administered to the provided patient prior to a planned start of CRRT. For example, for a patient starting on vancomycin after initiation of CRRT, a single loading dose of 10-20 mg/kg of total body weight may be administered to the patient intravenously over 60 minutes, after which the vancomycin may be added directly to the CRRT solution(s) according to embodiments disclosed herein. See step 56. Vancomycin may be added to the CRRT solution in vitro at a final concentration of 30 mg/L at CRRT initiation, regardless of age.

Alternatively, for a provided patient receiving vancomycin prior to the initiation of CRRT, if the last dose administered was less than about six to eight hours prior to CRRT initiation, no loading dose is administered to the patient intravenously; rather, the vancomycin is added only to the CRRT solution in vitro. See step 58. Otherwise, if the last dose was administered more than about six to eight hours prior to CRRT initiation, a loading dose of 10-20 mg/kg of total body weight is administered to the patient intravenously over 60 minutes, after which the vancomycin is added directly to the CRRT solution(s) according to embodiments disclosed herein. See step 60. Vancomycin may be added at a final concentration of 30 mg/L at CRRT initiation, regardless of age.

In step 62, a first serum vancomycin level is obtained from a blood sample from the patient about 8-12 hours after initiation of use of the CRRT solution mixed with vancomycin. All serum for vancomycin concentrations determination may be obtained directly from the blood sample from the patient provided via a central line, arterial line or peripheral venipuncture.

According to embodiments disclosed herein, drug concentrations in the solution(s) are adjusted in vitro based on an initial serum plateau level, and subsequent levels are thereafter obtained based on these changes. For instance, the target vancomycin plateau serum concentrations range is 15-30 mg/L. In step 64, a determination is made whether or not the serum level of the blood sample from the patient is within the target range. For instance, if the first vancomycin plateau level obtained is within range (see step 68), thereafter at least daily vancomycin plateau levels (or levels obtained at other specified time intervals) are obtained from blood samples from the patient for the duration of the procedure in which vancomycin is mixed in the solution. Alternatively, if the first vancomycin plateau level is not within range, the vancomycin concentration in the CRRT solution is adjusted in vitro, for instance, as discussed below. Subsequent vancomycin plateau levels may be obtained eight to twelve hours after each vancomycin concentration change and this process may continue until a therapeutic plateau level is obtained for a blood sample from the patient. See step 70. Once a therapeutic plateau level is obtained, daily vancomycin plateau levels may be obtained from blood samples from the patient for the duration the vancomycin is mixed in the CRRT solution.

If flow rates are changing, depending on the clinical scenario, the flow rates pertaining to the CRRT or dialysis prescription can be adjusted (increased or decreased) to effectively deliver a higher or lower amount of antibiotic (or other drug) separately or in addition to adjusting the concentration of the antibiotic in the CRRT solution bag(s). For instance, information concerning PK parameters based on the concentration of the antibiotic in the solution and the flow rates of the dialysate and/or replacement solution(s) may be used to determine a new concentration of vancomycin or like drug to be injected into the in-vitro CRRT solution. See step 72. Subsequent vancomycin plateau levels from blood samples from the patient may be obtained eight to twelve hours after each vancomycin concentration change and this process may continue until a therapeutic plateau level is obtained. See step 70. Once a therapeutic plateau level is obtained, at least daily vancomycin plateau levels may be obtained for the duration the vancomycin is mixed in the CRRT solution.

Based on the initial concentration of vancomycin in the CRRT solution, the flow rates, and the serum plateau level, adjustments to the vancomycin concentration in the CRRT solution may be conducted as follows:

Dose (mg/hr)=Concentration_{observed} (mg/L)*Clearance (L/hr)*PF, where PF is a penetration factor that describes the penetration of a drug, for instance such as an antibiotic, into a potential or actual site of infection such as the central nervous system (CNS), bone, or lung, as examples, and additionally takes into consideration the amount of protein binding of the respective drug in question.

The dose in mg/hr may include the amount of vancomycin from all of the flow rates from all sources of the CRRT solution. When the first serum plateau level is obtained, an equation can be solved to determine the clearance in L/hr. Once the clearance is determined, the desired steady state concentration (Cₛₛ) can be input and the new dose in mg/hr can be determined. Based on the flow rates and the dose (mg/hr) needed to obtain the desired Cₛₛ, the concentration needed in the CRRT solution to deliver the dose may be determined. Additionally, each of these PK parameters can be normalized by weight so that doses can be expressed as mg/kg/hr and clearances can be expressed as L/hr/kg, or another factor such as mL/min/kg.

Additional equations can also be used to determine pharmacokinetic parameters including elimination rate constant, Vd, half-life and CL. Such equations that could be utilized include: 1) ke=[ln (C1/C2)]/time difference; 2) t½=0.693/ke; 3) Vd=dose/initial concentration; 4) CL=ke*Vd; and 5) Dose (mg/kg/hr)=Css (mcg/mL)*CL, where CL=ke (hr⁻¹)*Vd (L/kg).

If the CRRT flow rates remain constant, the process for evaluating the serum plateau levels and potential changes to the vancomycin concentration (or any other drug) in the CRRT solution can be simplified as follows:

V_{jc}/V_{pobs})=(V_{jcn}/V_{pdes})

where V_{jc}=Vancomycin CRRT solution concentration,

V_{jcn}=New vancomycin CRRT solution concentration,

V_{pobs}=Observed vancomycin plateau level, and

V_{pdes}=Desired vancomycin plateau level.

Once the first vancomycin plateau level (V_{pobs}) is obtained, the vancomycin concentration in the CRRT solution is already known (V_{jc}). The desired or target vancomycin plateau level (V_{pdes}) can then be entered and the equation solved to determine the new vancomycin CRRT solution concentration (V_{jcn}).

The above referenced algorithms may be implemented in the form of a computer program, application (app), or other computer-readable format suitable for being executed on a computer, laptop, PC, tablet computer, smart phone or other electronic device, a part of a CRRT machine, a stand-alone unit, or the like. A user interface or the like may be provided with the above program or apparatus enabling ready use of the algorithms by the end user.

As discussed above, blood samples for vancomycin analysis may be obtained 8-12 hours after starting the CRRT solution mixed with vancomycin. Based on the first drug level obtained, changes to the CRRT solution concentration may be made to adjust to the desired serum concentration and serum levels from blood samples will be obtained 8-12 hours after each change to the vancomycin concentration in the CRRT solution. Once a therapeutic level is obtained, serum levels will be obtained as needed from blood samples, such as daily.

Upon mixing the vancomycin in the CRRT solution, the CRRT solution may be inspected for precipitation and crystallization for a period up to 10 minutes. Upon initiation of CRRT, the CRRT circuit may be inspected and evaluated on an hourly basis according to current standards of care. A part of the hourly standard of care inspection may involve inspection of the CRRT solution for precipitation and crystallization. This assessment should continue for the period of time the patient is receiving CRRT and vancomycin or other antibiotics is mixed in the CRRT solution.

The CRRT circuitry, including the filter, should also be inspected for clotting and adequate flow rates. Upon initiation of CRRT, the CRRT circuit may be inspected and evaluated on an hourly basis according to current standards of care for appropriate filter function and anticoagulation of the circuit which is included as a part of the hourly standard of care inspection. This assessment should continue for the period of time the patient is receiving CRRT and vancomycin or any other antibiotic is mixed in the CRRT solution. In addition, the patient is assessed daily for common adverse events including red man's syndrome in addition to serious adverse events, including mortality, for the period of time the patient is receiving CRRT with vancomycin mixed in the CRRT solution.

For patients receiving vancomycin, therapeutic drug monitoring currently occurs as part of routine clinical care. As such, serum samples for vancomycin concentration determination may continue to be sent for analysis as part of standard of care.

Blood samples for vancomycin concentration determination should be collected as part of normal standard of care. The timings of doses and sampling may be recorded, for instance, in military time.

Depending on the clinical scenario, patients are often started on multiple drugs or antibiotics (2-3 or more). Each drug or antibiotic can be added simultaneously to the CRRT solution bag (or similar dialysis solution), presuming they are stable in the same solution together, so that combinations (2 or more) of the drugs or antibiotics can be delivered at the same time. The starting concentration for each drug or antibiotic would be determined independently of the other. Once the CRRT treatment is started, serum concentrations may be obtained from blood samples at 8 to 12 hour time points for each antibiotic in the CRRT solution and then the above referenced dosing algorithm/app may be used to determine whether changes are required to be made to each antibiotic or drug independent or the others.

For purposes of adding an antibiotic to the dialysate or replacement solution, a flexible bag, such as a dialysate bag containing a fresh supply of dialysate solution, may be used. The bag may be provided with a port to which an adapter or vial interconnection device communicates and/or is integrally provided. The adapter may be configured to engage about a neck finish of a vial and to be positively locked thereto with a locking device or ring. The bag may also include additional ports, such as an output port through which the liquid solution exits the bag during a CRRT treatment.

Accordingly, when a medication or like substance, such as an antibiotic in powder or liquid form, is desired to be added to the liquid solution contained within the flexible bag, a user may grip the adapter in one hand and a vial having a stopper may be held in the opposite hand. The adapter may then be forced onto the neck finish of the vial thereby latching the adapter to the neck finish. Thereafter, a locking ring or the like may be advanced over the adapter to a locking position that reliably locks the adapter to the neck finish of the vial.

For purposes of mixing the liquid solution in the bag with the medication or like substance contained in the vial, the bag may be squeezed to force liquid solution contained within the bag into the vial. The vial may then be shaken to thoroughly mix the solution with the medicine and then the mixture may be caused to flow into the bag via forces of suction created by releasing any squeezing of the bag and/or by gravity by positioning the vial above the bag. After the mixture has been received within the bag, the bag is ready for use. The vial may remain attached to the bag during CRRT treatment for safety purposes providing a clear indication as to the fact that the contents of the vial have already been added to the bag. Alternatively, the vial may be removed from the bag and the adapter sealed close.

### Experimental Study

In this study, CRRT was performed using a Prismaflex System machine (Gambro, Lakewood, Colo.). The mode of dialysis, continuous venovenous hemofiltration or hemodiafiltration was dictated by an attending nephrologist prescribing the dialysis. The Prismaflex machine was initially primed with two 1-L bags of normal saline and 5,000 units of heparin. The machine was then primed with normal saline, 5% albumin, or a blood exchange (blood is transfused to the patient as the prime is wasted) depending on the percent extracorporeal volume of the patient with a dialysis solution utilizing HF1000, AN69 dialysis filters. Commercially available dialysate and replacement solutions were used for all CRRT patients. The CRRT circuit and solutions were inspected and observed for adverse events, such as specifically for precipitation and clotting. The CRRT solution(s) and circuit were visually inspected after addition of the CRRT circuit prior to the final connection to the patient.

Patient demographics for this study are provided in Table 1. Eleven patients receiving CRRT concurrently received vancomycin and CRRT, all of which were given as a CI in the CRRT solution. The median eGFR on ICU admission was 39.9 mL/min/1.73 m² (25th to 75th IQR, 25.8-53.4 mL/min/1.73 m²) compared with a median eGFR of 26.7 mL/min/1.73 m² (25th to 75th IQR, 18.8-42.7 mL/min/1.73 m²) with the initiation of CRRT. Four of the eleven patients (36%) had an “injury” pRIFLE classification at CRRT initiation, and eight of the eleven patients (64%) had a “failure” pRIFLE classification at CRRT initiation.

TABLE 1

| Patient No. | Gender | Age (Yr) | Diagnosis |
|---|---|---|---|
| 1 | Female | 0.08 | Necrotizing enterocolits |
| 2 | Female | 14 | Myocarditis, cardiogenic shock |
| 3 | Female | 10 | Influenza sepsis and respiratory |
| | | | failure |
| 4 | Female | 2 | Fever and neutropenia |
| 5 | Male | 15 | Septic shock |
| 6 | Female | 10 | End-stage renal disease |
| 7 | Male | 6 | Steven-Johnson syndrome |
| 8 | Female | 16 | Liver failure/shock |
| 9 | Male | 18 | Respiratory failure |
| 10 | Male | 0.08 | Sepsis |
| 11 | Male | 1 | Cardiac arrest |

ICU and hospital length of stay, duration of vancomycin, CRRT modality, and method of anticoagulation are presented in Table 2 for the eleven patients.

TABLE 2

| | ICU | Hospital | Vancomycin | Total | | | |
|---|---|---|---|---|---|---|---|
| | Length of | Length of | Duration | Vancomycin | CRRT | | |
| Patient | Stay | Stay | on CRRT | Duration | Duration | CRRT | |
| No. | (days) | (days) | (days) | (days) | (days) | Modality | Anti coagulation |
| 1 | 47 | 47 | 3 | 15 | 26 | CVVHD/ | Heparin/citrate |
| | | | | | | CVVH | |
| 2 | 8 | 17 | 2 | 4 | 5 | CVVH | Heparin |
| 3 | 11 | 11 | 3 | 6 | 8 | CVVHDF/ | Heparin/citrate |
| | | | | | | CVVH | |
| 4 | 16 | 39 | 2 | 2 | 10 | CVVH | Citrate |
| 5 | 9 | 24 | 3 | 3 | 6 | CVVHDF | Citrate |
| 6 | 19 | 28 | 2 | 4 | 5 | CVVH | Heparin |
| 7 | 52 | 52 | 25 | 32 | 42 | CVVHDF | Heparin |
| 8 | 20 | 20 | 7 | 8 | 12 | CVVH | Heparin |
| 9 | 9 | 9 | 2 | 6 | 2 | CVVHD | Heparin |
| 10 | 11 | 21 | 2 | 3 | 2 | CVVHDF | Heparin |
| 11 | 38 | 55 | 3 | 3 | 6 | CVVHDF/ | Citrate/Heparin/ |
| | | | | | | CVVH | citrate |
| Mean | 21.82 | 29.36 | 4.91 | 7.8 | 11.27 | | |
| SD | 16.14 | 16.33 | 6.82 | 8.8 | 12.17 | | |
| Min | 8.00 | 9.00 | 2.00 | 2 | 2.00 | | |
| Max | 52.00 | 55.00 | 25.00 | 32 | 42.00 | | |
| Median | 16.00 | 24.00 | 3.00 | 4 | 6.00 | | |
| IQR, 25 | 10 | 18.5 | 2 | 3 | 5 | | |
| IQR, 75 | 29 | 43 | 3 | 7 | 11 | | |

Description of vancomycin concentration in the CRRT solution(s) and resultant serum plateau concentrations are presented in Table 3. In Table 3, the first column corresponds to the patient number; the second column corresponds to the concentration of vancomycin added to the CRRT solution; and the third column displays the resulting serum plateau level obtained based on the CRRT solution listed in column 2.

TABLE 3

| | Vancomycin | |
|---|---|---|
| | CRRT solution | Vancomycin |
| Patient | Concentration | Level |
| No. | (μg/mL) | (μg/mL) |
| 1 | 18 | 18 |
| 1 | 18 | 17.6 |
| 1 | 18 | 17.4 |
| 2 | 18 | 23.6 |
| 2 | 18 | 22.6 |
| 2 | 18 | 19 |
| 3 | 30 | 28 |
| 3 | 25 | 26.4 |
| 3 | 25 | 24 |
| 4 | 25 | 21.6 |
| 4 | 25 | 22.3 |
| 5 | 30 | 18.9 |
| 5 | 30 | 21.4 |
| 5 | 30 | 20.1 |
| 6 | 30 | 26.3 |
| 6 | 25 | 24.7 |
| 7 | 30 | 12.9 |
| 7 | 35 | 23.5 |
| 7 | 35 | 23 |
| 7 | 35 | 26.8 |
| 7 | 18 | 20.9 |
| 7 | 20 | 19.8 |
| 7 | 20 | 20.5 |
| 8 | 30 | 21.2 |
| 8 | 25 | 26.4 |
| 8 | 25 | 25 |
| 8 | 25 | 26 |
| 8 | 25 | 22.8 |
| 8 | 25 | 23.2 |
| 8 | 25 | 21.7 |
| 9 | 25 | 28.7 |
| 9 | 25 | 26.3 |
| 10 | 30 | 20.9 |
| 10 | 30 | 21.4 |
| 11 | 30 | 20.4 |
| 11 | 30 | 20.8 |
| 11 | 30 | 20.4 |
| Mean | 26.5 | 22.3 |
| SD | 5 | 3.3 |
| Min | 18 | 12.9 |
| Max | 35 | 28.7 |
| Median | 25 | 21.8 |
| IQR, 25 | 25 | 20.9 |
| IQR, 75 | 30 | 25 |

The CRRT modality, dose, and flow rate information are presented in Table 4. All patients received a loading dose prior to the initiation of CRRT.

TABLE 4

| | | | | | Total | Pre Blood | Post Blood | Dialysate |
|---|---|---|---|---|---|---|---|---|
| | | | | Blood | Replacement | Pump | Pump | Flow |
| Patient | Age | Weight | CRRT | Flow | Flow | Replacement | Replacement | Rate |
| No. | (yr) | (kg) | Modality | (mL/min) | (mL/min) | Rate (mL/hr) | Rate (mL/hr) | (mL/hr) |
| 1 | 0.08 | 3.1 | CVVHD | 700 | 150 | 100 | 50 | 200 |
| | | | CVVH | 50 | 300 | 250 | 50 | N/A |
| | | | CVVH | 70 | 200 | 100 | 50 | N/A |
| 2 | 14 | 61.7 | CVVH | 130 | 2000 | 1950 | 100 | N/A |
| | | | CVVH | 170 | 1500 | 1400 | 100 | N/A |
| | | | CVVH | 170 | 2000 | 1900 | 100 | N/A |
| 3 | 10 | 33.6 | CVVHDF | 100 | 750 | 250 | 500 | 1500 |
| | | | CVVHDF | 100 | 750 | 750 | N/A | 1500 |
| | | | CVVH | 100 | 750 | 750 | N/A | N/A |
| | | | CVVH | 100 | 1200 | 1200 | N/A | N/A |
| | | | CVVH | 100 | 1000 | 800 | 200 | N/A |
| 4 | 2 | 15.2 | CVVH | 120 | 750 | 500 | 250 | N/A |
| | | | CVVH | 90 | 700 | 400 | 300 | N/A |
| | | | CVVH | 100 | 500 | 300 | 200 | N/A |
| | | | CVVH | 800 | 500 | 300 | 200 | N/A |
| | | | CVVH | 800 | 400 | 200 | 200 | N/A |
| | | | CVVHDF | 80 | 400 | 200 | 200 | 1000 |
| 5 | 15 | 65 | CVVHDF | 180 | 1000 | 950 | 50 | 1000 |
| | | | CVVHDF | 150 | 1500 | 1250 | 250 | 500 |
| 6 | 10 | 24 | CVVHDF | 80 | 500 | 450 | 50 | 500 |
| 7 | 6 | 21 | CVVHDF | 80 | 500 | 450 | 50 | 500 |
| | | | CVVHDF | 80 | 600 | 500 | 100 | 500 |
| 8 | 16 | 54.4 | CVVHDF | 100 | 1000 | 750 | 250 | 800 |
| | | | CVVHDF | 100 | 1250 | 1000 | 250 | 1000 |
| | | | CVVHDF | 200 | 1250 | 1000 | 250 | 800 |
| | | | CVVHDF | 200 | 2000 | 1500 | 500 | 800 |
| | | | CVVHDF | 250 | 2250 | 1750 | 500 | 800 |
| | | | CVVHDF | 250 | 2500 | 2000 | 500 | 1000 |
| | | | CVVHDF | 200 | 3000 | 2500 | 500 | 1000 |
| | | | CVVHDF | 2000 | 2050 | 2000 | 50 | 1000 |
| 9 | 18 | 58.1 | CVVHD | 180 | N/A | N/A | N/A | 2000 |
| 10 | 0.08 | 4.2 | CVVHDF | 40 | 600 | 550 | 50 | 400 |
| | | | CVVHDF | 50 | 600 | 550 | 50 | 400 |
| 11 | 1 | 14.87 | CVVHDF | 60 | 500 | 450 | 50 | 500 |
| | | | CVVHDF | 60 | 800 | 700 | 100 | 500 |

The median (25th to 75th IQR) concentration of vancomycin added to the dialysate solution(s) was 25 mg/L (25-30 mg/L). The median (25th to 75th IQR) vancomycin serum plateau concentration was 21.8 (20.9-25). Ten of the eleven patients (91%) achieved a therapeutic serum vancomycin level within 8 hours of starting the mixing of vancomycin into the CRRT solution. The serum level for one patient with a vancomycin serum plateau level less than 15 mg/L at 8 hours was 12.9 mg/L, and this patient achieved a therapeutic level of 23.5 mg/L within 16 hours of starting CRRT after the vancomycin concentration in the CRRT solution was adjusted. Regardless of the CRRT modality, dose, and flow rate, all patients achieved therapeutic vancomycin serum plateau levels. On mixing the vancomycin in the CRRT solution, no adverse events were observed regarding the precipitation of vancomycin with the CRRT solution(s) or CRRT circuit.

Only two patients, patients 4 and 9, had cultures that were positive for a gram-positive organism. Patient 4 had Streptococcus pneumoniae isolated from pleural fluid with the initiation of the empiric antimicrobial regimen, including vancomycin. The vancomycin was continued for a period of 2 days for patient 4. Subsequent cultures obtained from the blood and the respiratory system were negative. The isolate of Streptococcus pneumoniae was penicillin sensitive, and the patient was changed to ampicillin for the remainder of the treatment course. Patient 9 had methicillin-sensitive Staphylococcus aureus isolated from a respiratory culture within 24 hours of starting vancomycin. Patient 9 was continued on vancomycin for a period of 6 days. On day 6 of vancomycin therapy, the repeat respiratory culture was negative, in addition to the blood culture being negative during that time. After the 6 days of vancomycin therapy, the antimicrobial regimen was narrowed, and vancomycin therapy was discontinued.

Adult data suggests that vancomycin pharmacokinetics observed with IV dosing is variable and is affected by factors such as inotropes/vasopressors, extrarenal clearance of vancomycin, CRRT intensity, CRRT circuitry, and even albumin concentrations. (See DelDot M E, Lipman J, Tett S E: Vancomycin pharmacokinetics in critically ill patients receiving continuous venovenous haemodiafiltration, Br J Clin Pharmacol 2004; 58:259-268, and Bressolle F, Kinowski J M, de la Coussaye J E, et al: Clinical pharmacokinetics during continuous haemofiltration. Clin Pharmacokinet 1994; 26:457-471.) Earlier adult studies demonstrated reduced vancomycin and drug clearance in general, but the intensity of CRRT in those investigations was lower than with the current, contemporary CRRT devices and modalities. (See Bressolle F, Kinowski J M, de la Coussaye J E, et al: Clinical pharmacokinetics during continuous haemofiltration. Clin Pharmacokinet 1994; 26:457-471.) For example, one reference (Joy M S, Matzke G R, Frye R F, et al: Determinants of vancomycin clearance by continuous venovenous hemofiltration and continuous venovenous hemodialysis, Am J Kidney Dis 1998; 31:1019-1027) demonstrated that escalation in CRRT intensity from hemofiltration to hemodiafiltration increased vancomycin clearance by greater than or equal to 30%, mirroring data for other hydrophilic drugs such as β-lactams. Furthermore, another (Covajes C, Scolletta S, Penaccini L, et al: Continuous infusion of vancomycin in septic patients receiving continuous renal replacement therapy, Int J Antimicrob Agents 2013; 41:261-266) reported the ultrafiltration rate to be a significant determinant of vancomycin concentrations in a cohort of critically ill adults. As such, no clear guidance or recommendation regarding the optimal vancomycin dosing regimen in adult or pediatric CRRT exists.

Both vancomycin and β-lactams display time-dependent bactericidal activity, meaning that the extent of bacteria killing correlates with the time that the drug concentration remains above the MIC at the site of infection. Therefore, a CI dosing modality is an attractive option for vancomycin and the β-lactams. CI can provide benefits compared with an intermittent infusion, considering that target concentrations are obtained sooner. CI has been shown to improve outcomes with β-lactams; CI vancomycin has demonstrated similar efficacy with fewer adverse events. As it may be cumbersome to calculate AUC, many clinicians use trough concentrations as a surrogate to estimate AUC. This is especially true in pediatrics, since the target AUC/MIC greater than or equal to 400:1 target used in adults has not been validated in pediatrics. When using CI vancomycin, a target plateau concentration ranging from 15-20 mg/L, and up to 30 mg/L may be appropriate depending on the MIC of the infecting organism. Data regarding CI vancomycin in pediatrics range from neonates to adolescents. Depending on the age of the patient and comorbidities, dosing for CI vancomycin in neonates and pediatric patients range from 20 to 60 mg/kg/day and resulted in the target trough concentration being achieved. By mixing the vancomycin into the CRRT solution, 10 of 11 patients (91%) achieved the target vancomycin plateau level within 8 hours of starting CRRT.

Although there are different CRRT modalities, in general, CRRT relies on the principle of solute movement across a semipermeable membrane and a positive hydrostatic pressure forces water and solutes across the filter membrane from the blood compartment to the filtrate compartment. In addition, replacement fluid is typically used to replace fluid volume and electrolytes. Depending on the indication for CRRT, different modalities, membranes, and doses or intensities can be used. One advantage of adding vancomycin into the CRRT solution(s) is the relative safety regarding ensuring the delivery of vancomycin to the patient while they are receiving CRRT and to the patient not receiving additional vancomycin while they are not receiving CRRT. Patients can be disconnected from the CRRT circuit during their CRRT run, due to many different reasons, such as circuit changes, traveling for tests and even for procedures. The addition of vancomycin to the CRRT solution(s) for administration minimizes the chance of elevated serum concentrations and the potential for toxicities related to elevated serum concentrations.

As the amount of vancomycin added to the CRRT solution is less than or equal to 30 mg/L, and unlike intermittent dosing where higher doses are used with anticipation of enhanced elimination with CRRT, unplanned interruptions of CRRT are less likely to lead to elevated serum concentrations. Avoiding this minimizes the risk of exposing end organs to prolonged periods of elevated serum concentrations, which is a presumed risk factor for nephrotoxicity and potentially ototoxicity. Furthermore, by the addition of vancomycin to the CRRT solution(s), the vancomycin serum plateau level should correlate closely with the vancomycin concentration in the CRRT solution(s) regardless of the membrane, CRRT modality, or CRRT intensity/dose.

Finally, there is a potential cost saving by using this method. The addition of vancomycin to the CRRT solution(s) should result in less total vancomycin used per day. For example, if the concentration is 30 mg/L and three 3-L bags are being used, the total vancomycin used would be 270 mg. If a total of six bags are used per day, the total vancomycin per day would be 540 mg. A 10-kg patient started on 60 mg/kg/day would use 600 total mg of vancomycin per day. The costs associated with checking levels should be both reduced and minimized because new pharmacokinetics should not have to be determined if the CRRT modality or intensity/dose is changed.

According to the study disclosed herein, the addition of vancomycin to the dialysate solution(s) is an effective modality that is used for delivering vancomycin CI and for ensuring therapeutic vancomycin serum concentrations in the setting of pediatric CRRT as well as adult CRRT.

While the principles of the invention have been described above in connection with specific devices, systems, and/or methods, it is to be clearly understood that this description is made only by way of example and not as limitation. One of ordinary skill in the art will appreciate that various modifications and changes can be made without departing from the scope of the claims below. Accordingly, the specification and figures are to be regarded in an illustrative rather than a restrictive sense, and all such modifications are intended to be included within the scope of the present invention.

We claim:

1. A method of preparing solution for use during a dialysis, hemodialysis, or Continuous Renal Replacement Therapy (CRRT) treatment of a patient, comprising the steps of:
adding a drug to a first CRRT replacement or dialysate solution contained within a first flexible bag so that the first solution within the first flexible bag contains a first predetermined concentration of the drug;
wherein the drug is an antibiotic, vancomycin, a β-lactum antibiotic, glycopeptide, oxazolidinone, an anti-infective that displays time dependent anti-infective properties, an anti-viral agent, an anti-fungal agent an analgesic, a sedative, an anti-epileptic, a vasoactive, or an anti-hypertensive; and
wherein said step of adding the drug to the first solution in the first flexible bag uses an adapter integrally formed with the first flexible bag configured to engage about a neck finish of a vial and to be positively locked thereto with a locking device of the adapter and comprises the steps of:
connecting the adapter of the first flexible bag containing the first solution to a separate first vial containing the drug;
after said connecting step, securing the locking device of the adapter in a locking position to lock the adapter to the first vial;
after said securing step, squeezing the first flexible bag to cause an amount of the first solution contained in the first flexible bag to pass through a channel extending through the adapter and into the first vial;
mixing the solution and drug within the first vial by shaking the first vial to form a mixture; and
causing the mixture to pass through the adapter and into the first flexible bag.

2. The method according to claim 1, further comprising the step of:
adding a drug to a second CRRT replacement or dialysate solution contained within a second flexible bag so that the second solution in the second flexible bag contains a second predetermined concentration of the drug such that the first solution and the drug in the first solution are the same as the second solution and the drug in second solution except that the second predetermined concentration is different from the first predetermined concentration;
wherein said step of adding the drug to the second solution in the second flexible bag comprises the steps of:
connecting an adapter of the second flexible bag containing the second solution to a separate second vial containing the drug;
after said connecting step, securing a locking device of the adapter in a locking position to lock the adapter of the second flexible bag to the second vial connected thereto;
after said securing step, squeezing the second flexible bag to cause an amount of the second solution contained in the second flexible bag to pass through a channel extending through the adapter of the second flexible bag and into the second vial;
mixing the solution and drug within the second vial by shaking the second vial to form a mixture; and
causing the mixture to pass through the adapter of the second flexible bag and into the second flexible bag.

3. A method of preparing solutions for continuous renal replacement therapy (CRRT), comprising the steps of:
adding an antibiotic to a first solution provided for continuous renal replacement therapy (CRRT) such that the first solution contains a first predetermined concentration of the antibiotic; and
adding an antibiotic to a second solution provided for continuous renal replacement therapy (CRRT) such that the second solution contains a second predetermined concentration of the antibiotic such that the antibiotic in the first solution is the same as the antibiotic in the second solution and such that the second predetermined concentration is different from the first predetermined concentration;
wherein the first solution and the second solution are contained within separate flexible bags and are a CRRT replacement solution or a CRRT dialysate solution; and
wherein the antibiotic is vancomycin, a β-lactum antibiotic, glycopeptide, oxazolidinone, or an anti-infective that displays time dependent anti-infective properties.

4. The method according to claim 3, wherein the antibiotic is vancomycin.

5. A method of administering an anti-infective, comprising the steps of:
adding an antibiotic to a solution;
after said adding step, performing continuous renal replacement therapy (CRRT) treatment;
wherein the CRRT treatment is selected from continuous veno-venous hemofiltration (CVVH), continuous veno-venous hemodialysis (CVVHD), and continuous veno-venous hemodiafiltration (CVVHDF);
wherein the solution is a CRRT replacement solution or a CRRT dialysate solution; and
wherein the antibiotic is vancomycin, a β-lactum antibiotic, glycopeptide, oxazolidinone, or an anti-infective that displays time dependent anti-infective properties.

6. The method according to claim 5, wherein the antibiotic is vancomycin.
